(19) United States
(12) Patent Application Publication
(10) Pub. No.: US 2013/0006135 A1
(43) Pub. Date: Jan. 3, 2013

(54) PROCESS FOR MANUFACTURING AN ELECTRODE FOR MEDICAL USE AND ELECTRODE OBTAINED BY THE IMPLEMENTATION OF THIS PROCESS

(75) Inventor: Christophe Boillon, Pouligney (FR)

(73) Assignee: DIXI MICROTECHNIQUES, Besancon Cedex (FR)

(21) Appl. No.: 13/583,663

(22) PCT Filed: Mar. 15, 2011
(86) PCT No.: PCT/FR11/00141

§ 371 Date: Sep. 10, 2012

(30) Foreign Application Priority Data
Mar. 18, 2010 (FR) 10/51942

(51) Int. Cl. A61B 5/0476; A61N 1/05; B05D 1/32

(52) U.S. Cl. 600/544; 427/2.11; 607/116

(57) ABSTRACT

A process for manufacturing an electrode for medical use and electrode obtained by the implementation of this process. The manufacturing process, for manufacturing the electrode for medical use, such as a cortical electrode (1) intended for use at brain level, comprises the steps of using a silicone strip (3) to form a flexible substrate (30), placing, on the flexible substrate, a mask (5) determining a pattern (6) arranged to define at least one electrical track (2) having at least one contact pad (20), and depositing a metal layer on the flexible substrate (30) through the mask (5) by a physical vapor deposition technique.

This application is a National Stage completion of PCT/FR2011/000141 filed Mar. 15, 2011, which claims priority from French patent application serial no. 10/51942 filed Mar. 18, 2010.

### FIELD OF THE INVENTION

The present invention relates to a process for manufacturing an electrode for medical use, such as a cortical electrode intended for use at brain level.

The present invention also relates to an electrode obtained by the implementation of the present process.

### PRIOR ART

Cortical electrodes are devices used, depending on the cases, for diagnosis purposes, for therapeutic purposes, or to carry out studies. They are thus currently used for recording electroencephalograms, for example in order to locate brain dysfunctions and make a preoperative diagnosis of medically-refractory epilepsies, or in order to obtain a neurophysiological mapping, during neurosurgical interventions. They are also widely used for performing direct intracerebral stimulation, which shows to be beneficial in certain pathologies such as pain syndromes, or intended for triggering auras, or other seizures, for observation purposes.

Depending on the case, these electrodes are simply arranged at scalp level, or placed directly in contact with the brain, through openings made in the skull.

An example of cortical electrodes available at present on the market comes in the shape of metallic pads out of platinum/iridium connected through electrical wires, also made of platinum/iridium, with suitable electrical recording or stimulation devices. These pads, connected each to an electrical wire, are arranged on a slender and flexible support, so that they define a grid able to fit perfectly the shape of the areas to be explored. Such a grid has a variable configuration, and it can in particular be pre-cut so as to provide the number of electrical contacts suitable for the surface of the concerned brain area.

A manufacturing process of such a grid of cortical electrodes consists, in a first phase, in arranging the metallic pads on a template, connecting them individually by welding with the electrical wires previously introduced in a silicone sheath and, in a second phase, in placing the pads-electrical wires set in a sandwich structure between two silicone sheets bonded together subsequently and cut out to obtain the final shape of the grid.

Such a process is not totally satisfactory and shows to be tedious and costly because of the high number of steps it involves, in particular for connecting each pad with an electrical wire. Furthermore, the raw materials used to manufacture the pads and the electrical wires, that is to say a platinum/iridium alloy, are also known for their high price, which in the end ineluctably affects the price of the cortical electrodes themselves.

Document U.S. Pat. No. 6,624,510 describes on the other hand another example of a cortical electrode comprising a flexible substrate, preferably a polyimide layer, on which layers of one or several metals are deposited by vacuum evaporation or electrolytic deposition. Each electrode comprises a contact pad out of platinum connected via a connection area with a recording device through an electrical track made of a titanium layer covered with a gold layer. The whole, except for the contact pads and the connection area, is covered with an electrically insulating film such as a silicone film. The manufacturing process described in this document is not totally satisfactory, since it requires a high number of steps difficult to carry out and it is based on the use of expensive materials.

Publication US 2007/0007240, the subject of which is a manufacturing process of an intracortical microelectrode provided with a flexible connector, is based on the CMOS semiconductors manufacturing technique. An embodiment variant of this process involves the realization of a connector on a semiconductor substrate by depositing a conductive layer that may include gold on a silicon substrate, covering the connector with a polymer layer that is vapor-deposited through a mask, then immersing the semiconductor substrate and the connector covered with the polymer layer in an etching bath, and removing the semiconductor substrate from the connector manufactured this way so that it becomes flexible. Such a process has the disadvantage of being complex and costly, since it requires many steps and many masks. It also uses liquid or solid-phase etching processes.

Another process described in publication US 2007/0005112 creates a connection between an electronic unit and a cortical electrode by depositing by galvanoplasty a biocompatible metal such as platinum or gold on a polyimide substrate that has no elasticity that would allow it to fit the round shapes of a skull.

### DESCRIPTION OF THE INVENTION

The present invention aims to remedy these disadvantages by offering a simplified process for manufacturing a cortical electrode, involving a limited number of operations and steps and based on the use of less costly materials, while this process may easily be industrialized.

To that purpose, the invention relates to a process of the kind stated in the preamble, in which one uses a silicone strip to form a flexible substrate, one places on said flexible substrate a mask that determines a pattern arranged to define at least one electrical track having at least one contact pad, and one deposits a metal layer on said flexible substrate through said mask by means of a physical vapor deposition technique.

According to a characteristic of this process, one uses a silicone strip of the type having a reinforced structure, stiffened by depositing a layer of a polymer on at least one of its sides.

According to another characteristic of the present process, one uses for the mask a sheet of a metal or of an alloy of metals chosen in the group including molybdenum, stainless steel, nickel, with a thickness preferably included between 50 μm and 200 μm.

Advantageously, one arranges a magnetized part on the side of the substrate opposite to the side on which said mask is applied, in order to achieve tightness between said substrate and said mask.

Furthermore, said process is characterized also in that one activates chemically the area of the flexible substrate that is not covered by said mask.

In this case, in order to activate said silicone strip, one subjects it to an ionic cleaning step carried out by means of a mix of oxygen and argon in the plasma state, and one then deposits a layer of titanium on it.

According to another characteristic of the process according to the invention, the metal used to define said electrical track is a noble metal or an alloy of noble metals.

An additional characteristic of the present process also provides that one covers the set formed by said flexible substrate and said electrical track, except for the contact pad, with a layer of a protective material, deposited through a second mask by means of a chemical vapor deposition technique.

The invention also relates to an electrode for medical use, obtained by the implementation of the process described previously, such as a cortical electrode intended to be used at brain level, said electrode comprising a silicone strip forming a flexible substrate, on which at least one metal layer, arranged to define at least one electrical track having at least one contact pad, is deposited.

According to a preferred embodiment, the silicone strip used has a reinforced structure, and preferably a thickness of at least 200 μm.

Moreover, according to the invention, said silicone strip is covered on at least one of its sides with a layer of a stiffening polymer.

In this case, said polymer is parylene, whose thickness has a value included between 0.5 μm and 10 μm.

Furthermore, the electrode according to the invention is also characterized in that the metal that defines at least one electrical track having at least one contact pad is a noble metal or an alloy of noble metals.

Preferably, the metal layer has a thickness of at least 400 nm.

An additional characteristic of the present invention is also defined by the fact that said electrode is covered, except for the contact pads, with a layer of a protective material, for example parylene having preferably a thickness of at least 1 μm.

### BRIEF DESCRIPTION OF THE DRAWINGS

The present invention and its advantages will be better revealed in the following description of an embodiment given as a non limiting example, in reference to the drawings in appendix, in which:

FIG. 1 is a cross-sectional view of an electrode according to the invention in the course of manufacture, according to section plane AA of FIG. 3,

FIG. 2 is a cross-sectional view of the electrode represented in FIG. 1, finalized, according to section plane AA of FIG. 3, and

FIG. 3 is a top view of an embodiment example of an electrode according to the invention.

### ILLUSTRATIONS OF THE INVENTION AND BEST WAY OF REALIZING IT

Referring to the figures, the present invention relates to a process for manufacturing a cortical electrode 1, consisting in depositing a layer of a metal on a flexible substrate made of a silicone strip 3 in order to define at least one conductive track 2, having at least one contact pad 20.

Silicone is a supple, flexible and biocompatible material that is advantageously able to fit a spherical shape and that has a hydrophilic property allowing a good adherence on the surface of the cerebral cortex.

The silicone chosen for the implementation of the present process is preferably of the type having a reinforced structure, for example by means of the insertion of a polyester textile mesh during its extrusion. It is furthermore provided to use a silicone strip 3 having preferably a thickness of at least 200 μm, for example 300 μm, stiffened by the application of a layer 4 of a biocompatible polymer such as for example parylene, arranged to cancel at least partly the elasticity of silicone. Of course, any other biocompatible polymer able to stiffen and smooth the surface of the silicone could be used with the same purpose of cancelling at least partly the elasticity of silicone in order to avoid any risk of microcut or breakage of the deposited conductive tracks.

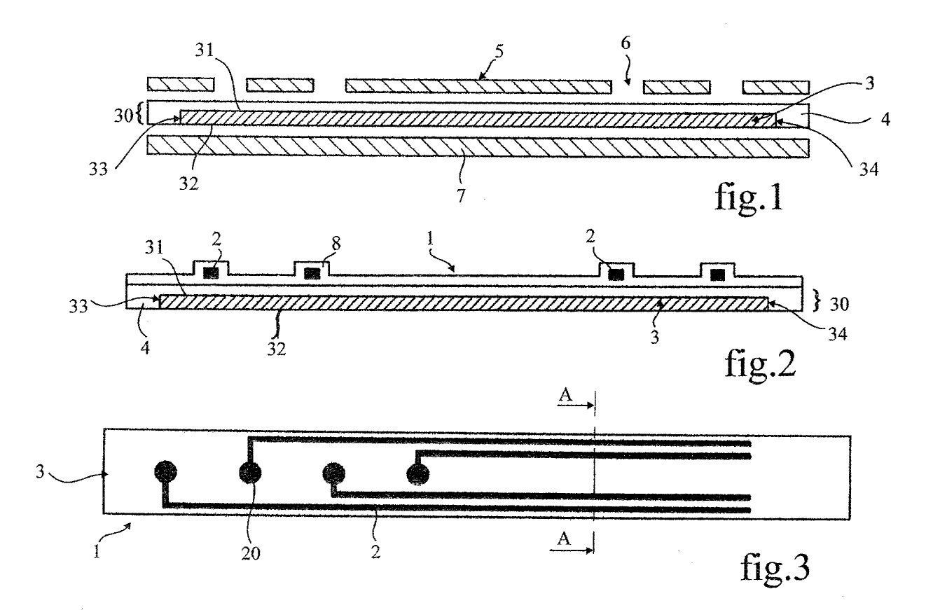

In compliance with the present process, the parylene layer 4 is applied, for example by chemical vapor deposition, on a thickness included for example between 0.5 μm and 10 μm, preferably 1 μm, so as to cover at least the edges 33, 34 of the silicone strip 3 (see FIGS. 1 and 2) and the side 31 intended to carry said conductive track 2.

A mask 5, bearing a pattern 6 arranged to define, in the represented example, a plurality of electrical tracks 2 having each at least one contact pad 20, is then located on side 31 of the flexible substrate 30 defined by the silicone strip 3 and the parylene layer 4.

According to the invention, this mask 5 is manufactured from a sheet of a metal or of an alloy of metals, chosen in the group including molybdenum, stainless steel, nickel or similar metals, this sheet having a thickness included for example between 50 μm and 200 μm. The pattern 6 is produced in the mask 5 by cutting said sheet by means of laser engraving or by using any other similar technique.

Preferably, and in order to achieve perfect tightness between the substrate 30 and the mask 5, one uses a mask 5 made from a sheet comprising nickel, and one places a magnetized plate 7 against the other side 32 of said substrate 30 to press the mask 5 on the substrate 30.

The set obtained this way is then placed in an enclosure arranged to carry out a physical vapor deposition of a layer of at least one metal on said substrate 30, through the pattern 6 of mask 5.

Prior to the step consisting in carrying out said metal deposition, the present process also recommends to activate chemically the area of the substrate 30 that is not covered by said mask 5, that is to say the area corresponding to pattern 6. This goal is achieved by performing an ionic cleaning by means of a mix of oxygen and argon, and by depositing then on said area a titanium layer with preferably a thickness of at least 400 nm. The titanium layer has the advantage of improving the adherence of the metal layer on substrate 30.

Finally, a layer of noble metal, for example gold, preferably with a thickness of at least 400 nm is deposited on substrate 30, through the pattern 6 of mask 5, by means of a physical vapor deposition technique such as, for example, the magnetron sputtering technique.

Gold has the advantage of being non-oxidizing and thus suits for direct contact with the surface of the brain. It is furthermore characterized by a good conductivity, and moreover allows visualizing the conductive tracks, in particular by Magnetic Resonance Imaging (MRI) or X-rays. Nevertheless, it can of course be replaced with another noble metal having equivalent properties, such as platinum, iridium, rhodium and silver. In addition, an alloy of noble metals such as for example platinum iridium or electrum could also be suitable.

After having removed the magnet 7 and the mask 5, the present process also involves covering the set formed by said flexible substrate 30 and the electrical tracks 2, except for the contact pads 20, with a layer 8 of a protective material such as in particular parylene or any other material having similar protective or insulating properties.

This deposit of a parylene layer 8 is carried out for example by means of a chemical vapor deposition technique, through a second, non represented mask, arranged to cover the pads 20 and allow the exposure of said tracks 2. On the other hand, said layer 8 has preferably a thickness of at least 1 μm.

Advantageously, the process according to the invention also involves carrying out a sterilization of the cortical electrode 1 obtained this way, for example with ethylene oxide.

Possibilities for Industrial Application:

This description shows clearly that the reduced number of steps of the present process, as well as the materials used, allow reaching the goals defined, that is to say facilitate the manufacture of a cortical electrode and reduce its costs.

Considering its cost-effectiveness, the cortical electrode 1 obtained this way can be intended for single use, making its use entirely secure and eliminating the need for heavy and costly sterilization techniques.

Moreover, the present process is based on the implementation of techniques adapted for an industrialization of the production, and the materials used can be recycled, which is particularly advantageous, in particular regarding the metals.

The present invention is not restricted to the example of embodiment described, but extends to any modification and variant which is obvious to a person skilled in the art while remaining within the scope of the protection defined in the attached claims.

1-22. (canceled)

23. A process of manufacturing an electrode for medical use, such as a cortical electrode (1) intended for use at brain level, in which, the process comprising the steps of:
using a silicone strip (3) to form a flexible substrate (30),
stiffening the silicone strip (3) by depositing a layer (4) of a polymer on at least one of side thereof,
placing a first mask (5), determining a pattern (6) arranged to define at least one electrical track (2) having at least one contact pad (20), on the flexible substrate (30), with the first mask (5) being made from a sheet of a metal or of an alloy of metals,
arranging a magnetized part (7) on the side (32) of the flexible substrate (30), opposite to the side on which the mask (5) is applied, in order to achieve tightness between the flexible substrate (30) and the first mask (5), and
depositing a metal layer on the flexible substrate (30) through the first mask (5) by a physical vapor deposition technique.

24. The process according to claim 23, further comprising the step of using a silicone strip (3) which has a reinforced structure.

25. The process according to claim 23, further comprising the step of using, as the first mask (5), a sheet of a metal or of an alloy of metals selected from the group consisting of molybdenum, stainless steel and nickel.

26. The process according to claim 25, further comprising the step of using a sheet of a metal or of an alloy of metals having a thickness between 50 μm and 200 μm.

27. The process according to claim 23, further comprising the step of, prior to the step of depositing the metal layer, chemically activating an area of the flexible substrate (30) that is not covered by the mask (5).

28. The process according to claim 27, further comprising the step of using, in order to activate the flexible substrate (30), subjecting the flexible substrate (30) to an ionic cleaning step carried out by a mixture of oxygen and argon in the plasma state, and
then depositing a layer of titanium thereon.

29. The process according to claim 23, further comprising the step of using a noble metal or an alloy of noble metals as the metal used to define the electrical track (2).

30. The process according to claim 23, further comprising the step of covering the flexible substrate (30) and the electrical track (2), except for the contact pad (20), with a layer (8) of a protective material deposited via a second mask by a chemical vapor deposition technique.

31. The process according to claim 23, further comprising the step of using parylene as the polymer forming the layer (4) or the protective material forming the layer (8).

32. An electrode (1) for medical use, such as a cortical electrode (1) intended to be used at brain level, obtained by implementation of a process comprising the steps of: using a silicone strip (3) to form a flexible substrate (30), stiffening the silicone strip (3) by depositing a layer (4) of a polymer on at least one of side thereof, placing a first mask (5), determining a pattern (6) arranged to define at least one electrical track (2) having at least one contact pad (20), on the flexible substrate (30), with the first mask (5) being made from a sheet of a metal or of an alloy of metals, arranging a magnetized part (7) on the side (32) of the flexible substrate (30), opposite to the side on which the mask (5) is applied, in order to achieve tightness between the flexible substrate (30) and the first mask (5), and depositing a metal layer on the flexible substrate (30) through the first mask (5) by a physical vapor deposition technique, wherein the electrode comprises:
a silicone strip (3) which forms a flexible substrate (30), on which at least one metal layer, arranged to define at least one electrical track (2) having at least one contact pad (20), is deposited, and
the silicone strip (3) is covered, on at least one of its sides, with a layer (4) of a stiffening polymer.

33. The electrode according to claim 32, wherein the silicone strip (3) used has a reinforced structure.

34. The electrode according to claim 33, wherein the silicone strip (3) has a thickness of at least 200 μm.

35. The electrode according to claim 32, wherein the polymer is parylene.

36. The electrode according to claim 35, wherein the thickness of the parylene is between 0.5 μm and 10 μm.

37. The electrode according to claim 32, wherein the metal is one of a noble metal and an alloy of a noble metals.

38. The electrode according to claim 37, wherein the metal layer has a thickness of at least 400 nm.

39. The electrode according to claim 32, wherein the electrode, except for the contact pads (20), is covered with a layer (8) of a protective material.

40. The electrode according to claim 39, wherein the protective material is parylene.

41. The electrode according to claim 40, wherein the parylene layer has a thickness of at least 1 μm.
